(19) United States
(12) Patent Application Publication
(10) Pub. No.: US 2023/0320716 A1
(43) Pub. Date: Oct. 12, 2023

(54) METHOD OF ENDOSCOPIC LESION TRACTION

(71) Applicant: Apollo Endosurgery US, Inc., Austin, TX (US)

(72) Inventors: Luis Amador, JR., Pflugerville, TX (US); Greg T. Prevatt, Austin, TX (US); Christopher John Gostout, Rochester, MN (US); Thomas Neudeck, Austin, TX (US)

(73) Assignee: Apollo Endosurgery US, Inc., Austin, TX (US)

(21) Appl. No.: 17/716,598

(22) Filed: Apr. 8, 2022

(51) Int. Cl. A61B 17/04

(52) U.S. Cl. CPC A61B 17/0401; A61B 2017/0464; A61B 2017/0496

(57) ABSTRACT

Endoscopic tissue traction includes advancing an anchor deployment system through a working channel of an endoscope positioned within a gastrointestinal tract and using the system to apply a first anchor having a length of suture fixed thereto into a distal portion of a tissue in a first wall in the body cavity. Then, the anchor deployment system is used to apply a like second anchor into a proximal portion of the tissue in the first wall, with the length of suture coupled to and displaceable relative to the second anchor. Next, the anchor deployment system is used to apply a like third anchor into a opposite second wall, with the length of suture coupled to and displaceable relative to the second anchor. The suture can then be tensioned relative to the second and third anchors to apply traction between the second wall and the relatively proximal portion of the tissue.

### CROSS-REFERENCE TO RELATED APPLICATION

This application is related to co-owned U.S. Ser. No. 16/701,276, filed Dec. 3, 2019, now published as US 2020/0178956, which is incorporated herein in its entirety.

### BACKGROUND OF THE INVENTION

### 1. Field of the Invention

The present methods relate to surgical procedures. More particularly, the methods are directed to applying traction to a lesion in an endoscopic procedure.

### 2. State of the Art

During endoscopic procedures in the gastrointestinal tract, and particularly the colon, it is often necessary to biopsy tissues because they have an appearance of a current disease state or prospective disease state that requires further histological evaluation. In addition, it is important to remove all of the tissue of interest and not leave diseased tissue at the margin of the removal.

For example, during endoscopic procedures in the gastrointestinal tract, suspect mucosal tissue is often removed in a procedure referred to endoscopic mucosal resection (EMR). To resect the target lesion, a lifting agent is injected in the submucosal tissue below the target lesion to form a bleb and elevate the lesion. Then, an electrosurgical instrument is used to cut the elevated lesion, and the cut lesion is retrieved, for example with a snare. One issue with the resection procedure is that it may leave diseased tissue at the margin, resulting is high lesion recurrence and which necessitates further therapeutic procedures.

In an alternate procedure of endoscopic submucosal dissection (ESD), a lesion in gastrointestinal tract is dissected by separating the mucosal and submucosal tissue layers. Fluid is injected into the submucosa, and an incision is created around the perimeter of the lesion. Then the lesion is carefully dissected from the deeper muscularis layers using electrocautery accessories and the lesion is removed as a single piece specimen. The procedure requires that the lesion be lifted while the fibers connecting the mucosa and deeper muscularis are cut. This technique allows for en bloc removal of large lesions.

Currently, surgeons use an endoscope with a cap attached at the end of the endoscope to aid in lifting the lesion during dissection. However, there is limited visualization in this operative system due to working very close to the tissue. Consequently, there is a high risk of tissue perforation and inadvertent dissection of blood vessels causing bleeding. In addition, because the dissection occurs so close to the end of the endoscope, the cap and dissection tool cannot effectively move independently.

### SUMMARY OF THE INVENTION

A method is provided for endoscopic tissue traction to facilitate endoscopic procedures.

A method is provided for endoscopic lesion traction to facilitate endoscopic tissue dissection.

A method is provided for endoscopic tissue dissection that permits the end of the endoscope to be located farther from the dissected tissue and increases visualization during the procedure.

In accord with the above, a method is provided for endoscopic tissue traction in a natural body cavity. The method includes providing a flexible endoscope. The endoscope is of a type well-known in the art and has a proximal end, a distal end, at least one working channel extending from the proximal end to the distal end, optical elements for surgical visualization and proximal controls for maneuvering and flexing the distal end of the endoscope.

In addition, the method includes providing a soft-tissue suture anchor deployment system. In an embodiment, the suture anchor deployment system includes a proximal handle, an elongate flexible delivery member having proximal and distal ends, the delivery member having a length and diameter sized for passage through the at least one working channel, the proximal end of the delivery member rotatably coupled to the handle, and a suture anchor engaging structure at the distal end of the delivery member.

In an embodiment, the anchor deployment system is separably loadable with individual anchors. In an embodiment, the anchors each include a helical portion that is adapted to engage tissue and be retained in the tissue. The suture anchor includes a distal helical portion and a proximal engagement structure for coupling with the anchor engagement structure at the distal end of the delivery member. In an embodiment, proximal engagement structure of the anchor is a receptacle portion. The suture anchor has a longitudinal axis that extends through the proximal engagement structure and helical portion. A suture eyelet is fixedly coupled to the suture anchor between the proximal and distal portions and is rotatable about the longitudinal axis of the suture anchor.

More particularly, in an embodiment the suture anchor receptacle includes a tubular member that has a retaining member adapted to engage with a post member of a deployment system such that when the post member of the deployment system is inserted into the receptacle the retaining member engages the post member so that the suture anchor is retained on the deployment system. The helical portion of the suture anchor is typically formed from a coiled wire or cut from tubing and having a sharpen tip such that when rotated the sharpened tip pierces and engages tissue. An elongate suture having proximal and distal ends extends through the suture eyelet such that rotation of the suture anchor by an attached deployment system causes the receptacle and helical portions to rotate without rotating the eyelet portion which keeps the elongate suture from winding on the deployment system and becoming entangled. In an embodiment, the suture is preloaded through multiple suture anchors, each of which is available for mounting at the distal end of the suture anchor deployment system.

In addition, the method includes providing an endoscopic needle injection device adapted to be inserted through a working channel of an endoscope to inject a fluid into tissue. Also, the method includes providing an endoscopic electrosurgical instrument adapted to be inserted through a working channel of an endoscope to cut tissue. The electrosurgical instrument may be a needle knife.

In accord with a method of the procedure, the endoscope is advanced into a natural body cavity through a natural orifice. In an embodiment, the endoscope is inserted through the rectum and into the colon. In another procedure, the endoscope is advanced through the mouth and into the stomach. In yet another procedure, the endoscope is advanced through the mouth, into the stomach, through the duodenum, and into the small intestine. In each of these body cavities formed by the gastrointestinal tract, the tract has an inner mucosal layer, an outer muscularis layer, connective fibers connecting the two layers and an outermost serosa layer.

The distal end of the needle injection device is advanced through a first working channel of the endoscope and inserted into a target tissue, such as a lesion. A lifting agent, such as saline, is injected under the target tissue to lift the target tissue and define a perimeter about the target tissue in a first wall of the gastrointestinal tract. The needle injection device is then removed from the first working channel of the endoscope.

The distal end of the electrosurgical instrument is then advanced through the first working channel of the endoscope and operated to cut through the mucosal tissue along the defined perimeter of the target tissue.

In an embodiment, the distal end of the suture anchor deployment system is then inserted through a second working channel. The suture anchor deployment system is advanced toward the distal portion of the target tissue, preferably adjacent the distal margin of the lesion, and operated to apply a first anchor thereat. A length of suture is fixed to the first anchor and extends out of the gastrointestinal tract. The suture anchor deployment system is then withdrawn from its working channel, the suture is thread through the suture eyelet of a second anchor, and the second anchor is mounted at the distal end of the suture anchor deployment system.

The suture anchor deployment system is again advanced through the working channel and advanced toward the proximal portion of the target tissue, preferably adjacent the proximal margin of the lesion, and operated to apply the second anchor thereat. The suture anchor deployment system is then withdrawn from the working channel, the suture is thread through the suture eyelet of a third anchor, and the third anchor is mounted at the distal end of the suture anchor deployment system.

The suture anchor deployment system is again advanced through the working channel and this time advanced toward a second wall of the gastrointestinal tract located opposite the first wall. Optionally, a similar process can be repeated to insert one or more additional suture anchors in the second wall. After the last suture anchor is implanted, the suture anchor deployment system is preferably withdrawn from the working channel of the endoscope. Then, the suture is tensioned from outside the patient through the working channel.

In another embodiment, utilizing an endoscope with a single working channel, the electrosurgical instrument is removed and then the suture anchor deployment system is inserted through the working channel and operated to deploy all tissue anchors. Then the suture anchor deployment system is removed from the working channel and an electrosurgical instrument is inserted into the working channel alongside the suture. The suture is tensioned from outside the patient through the working channel alongside the electrosurgical instrument.

In yet another embodiment using an endoscope with a single working channel, the procedure can be carried out as follows: the injection needle is inserted through the working channel and operated to inject fluid at the lesion to create a bleb; then, the injection needle is retracted from the working channel. The suture anchor deployment system is next inserted through the working channel, and then operated to position anchors in the lesion as well as in the opposing wall. Then, the suture anchor deployment system is removed from the endoscope, leaving the suture through the working channel. Next, the endoscope is removed from patient leaving the suture extending from the anchors and exiting the natural orifice. Then, the endoscope is again inserted through the natural orifice alongside the suture. An electrosurgical instrument can be inserted through the working channel. The suture can be tensioned from outside the patient to manipulate the lesion without interfering with the endoscope or the working channel.

When the suture is tensioned, such tension causes the suture, fixed to the first anchor but movable relative to the other anchors, to be drawn relative to the second and third anchors and thereby apply traction between the second wall and the relatively proximal portion of the target tissue, i.e., the proximal margin of the lesion. This stretches the connective fibers between the mucosal layer and the muscularis layer of the dissected lesion and allows for precise tissue manipulation.

The electrosurgical instrument is then operated to cut the stretched connective fibers under the proximal portion of the lesion. The second anchor lifts up toward the second wall. Further tensioning the suture draws the suture relative to the first and third anchors and thereby applies continued traction between the second wall and the relatively distal portion of the target tissue, i.e., distal margin of the lesion, to stretch connective fibers between the mucosal layer and the muscularis layer. The electrosurgical instrument is again operated to cut the stretched connective fibers under the distal margin of the lesion to separate the lesion from the muscularis layer. The process may be repeated in intermediate steps before the lesion is completely separated.

The suture and lesion are withdrawn from patient, along with the endoscope. The dissected tissue is retrieved en masse for histological examination.

The method permits controlled tension to be applied to a lesion even after the connective fibers under the proximal margin of the lesion are cut. The method also permits identifying and applying tension at multiple locations during removal of the lesion. This facilitates maintaining high visibility at the operative location during the procedure and permits clean and efficient removal of the entire lesion and can reduce the risk of bleeding or perforation.

### BRIEF DESCRIPTION OF THE DRAWINGS

FIG. 1 is a side elevation of an endoscopic suture anchor deployment system used in a method described herein, shown in a configuration prepared for delivery through a working channel of an endoscope.

FIG. 2 is an enlarged partially-transparent side elevation of the distal end of the endoscopic suture anchor deployment system of FIG. 1, shown in a configuration with a suture anchor exposed for deployment.

FIGS. 3 through 12 illustrate selected steps of a method of endoscopic tissue dissection described herein.

### DETAILED DESCRIPTION OF THE PREFERRED EMBODIMENTS

With reference to the following description, the terms “proximal” and “distal” are defined in reference to the hand of a user of the device, with the term “proximal” being closer to the user's hand, and the term “distal” being further from the user's hand such as to often be located further within a body of the patient during use. Further, in accord with a general description of the system and its exemplar use, described in more detail below, the system is provided and used to target tissue, deploy a suture anchor into tissue, and reconfigure the anchored tissue. Such targeting, fastening and reconfiguring are preferably, though not necessarily, performed in conjunction with a surgical scope, such as a laparoscope or an endoscope. In embodiments described herein, the steps may be used to reconfigure tissue through or with the aid of an endoscope in which the instrument acting to reconfigure the tissue are inserted through a natural orifice, namely the gastroesophageal pathway, preferably without incision to either the dermal or internal tissues of a patient in order to effect for passage of the required instruments. Specifically, it is recognized that piercing the tissue for insertion of a fastener does not effect an incision in the tissue.

In accord with an embodiment of a method for endoscopic tissue traction in a natural body cavity, an endoscope, an endoscopic needle injection device, an endoscopic electrosurgical cutter, and a soft-tissue suture anchor deployment system are all provided.

The endoscope is of a type well-known in the art and has a proximal end, a distal end, at least one working channel extending from the proximal to the distal end, optical elements for surgical visualization, and proximal controls for maneuvering and flexing the distal end of the endoscope.

The endoscopic injection needle includes a flexible catheter having a proximal end and a distal end and a lumen between the proximal and distal ends sufficient to extend through the working channel of the endoscope, a tissue penetrating needle at the distal end and in communication with the lumen, a fluid store at the proximal end of the catheter and communicable with the lumen, and a plunger capable of displacing the fluid from the fluid store through the lumen and out of the needle. Endoscopic injection needles of the type usable in the method are well-known in the art.

The endoscopic electrosurgical instrument includes a flexible catheter mounted with a cautery instrument, such as a needle knife, at its distal end. The flexible catheter has a length adapted to extend through the working channel of the endoscope and powered to cut tissue. Endoscopic electrosurgical instruments of the type usable in the method are well-known in the art.

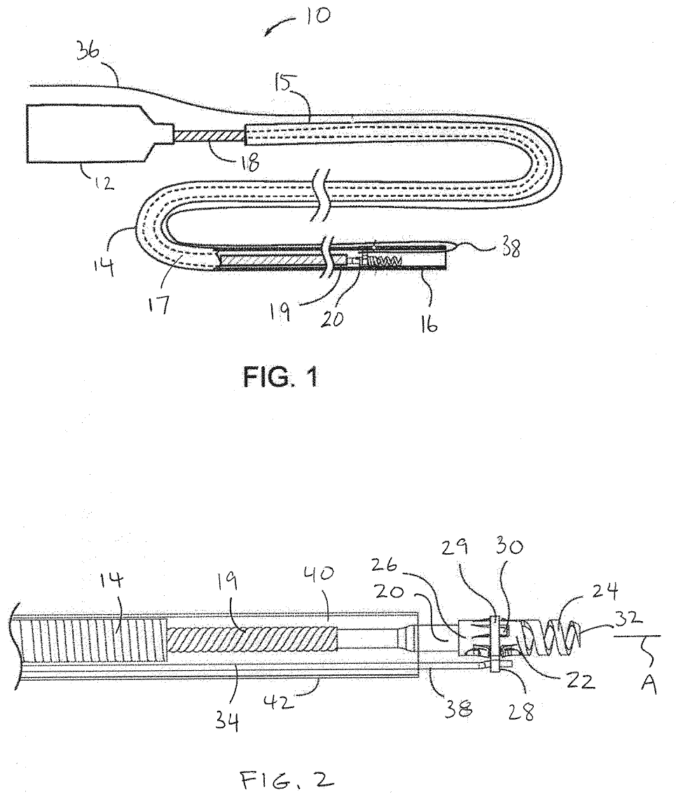

In addition, Referring to FIGS. 1 and 2, the method includes providing a soft-tissue suture anchor deployment system 10. In an embodiment, the suture anchor deployment system 10 includes a proximal handle 12, an outer sheath 14 having proximal and distal ends 15, 16, with the proximal end 14 fixed relative to the proximal handle 12, an elongate flexible delivery member 17 extending through the sheath 14 and having proximal and distal ends 18, 19, the delivery member 17 having a length sized to extend through the at least one working channel 40 of the endoscope 42, the proximal end 18 of the flexible delivery member 17 rotatably coupled to the handle 12, and a suture anchor engaging structure 20 at the distal end 19 of the delivery member.

The anchor deployment system 10 is separably loadable with individual anchors 22. In an embodiment, the anchors 22 each include a helical portion 24 that is adapted to engage tissue and be retained in the tissue. The suture anchor 22 includes a distal helical portion 24 and a proximal engagement structure 26 for coupling with the anchor engagement structure at the distal end 19 of the delivery member 17. In an embodiment, the proximal engagement structure 26 of the anchor is a receptacle portion. The suture anchor 22 has a longitudinal axis A that extends through the proximal engagement structure 26 and helical portion 24. A suture eyelet 28 is defined in a ring 29 coupled to the suture anchor 22 between the proximal and distal portions, with the ring rotatable about the longitudinal axis A of the suture anchor.

More particularly, in an embodiment the suture anchor receptacle 26 includes a tubular member that has a retaining member 30 adapted to engage with a post member 20 of a deployment system 10 such that when the post member 20 of the deployment system is inserted into the receptacle 26, the retaining member engages the post member so that the suture anchor is retained on the deployment system. The helical portion 24 of the suture anchor is typically formed from a coiled wire or from laser cut tubing having a sharpen tip 32 such that when rotated the sharpened tip pierces and engages tissue. An elongate suture 34 having proximal and distal ends 36, 38 extends through the suture eyelet 28 such that rotation of the suture anchor by an attached deployment system causes the receptacle 26 and helical portions 24 to rotate without rotating the eyelet portion 28 which keeps the elongate suture 34 from winding on the deployment system and becoming entangled. Such an anchor deployment system and anchors are described in detail in co-owned US Pub. No. US 2020/00178956, previously incorporated herein by reference.

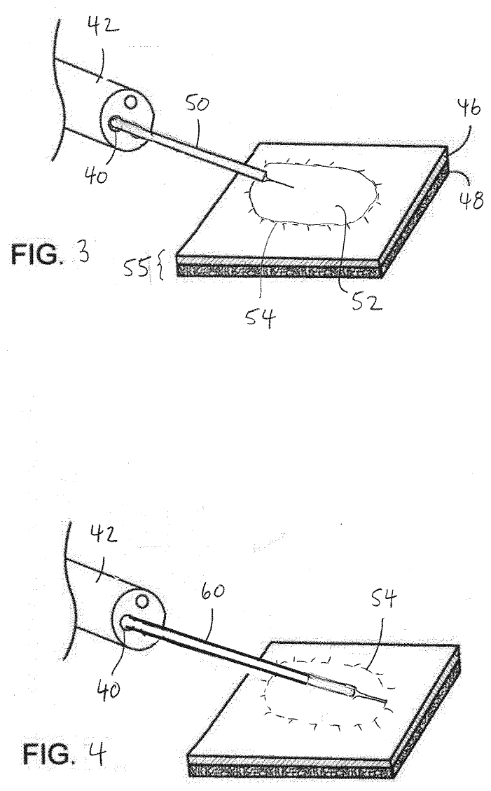

In accord with a method of the procedure, the endoscope 42 is advanced into a natural body cavity through a natural orifice. In an embodiment, the endoscope is inserted through the rectum and into the colon. In another procedure, the endoscope is advanced through the mouth and into the stomach. In yet another procedure, the endoscope is advanced through the mouth, into the stomach, through the duodenum, and into the small intestine. Referring to FIG. 3, in each of the body cavities formed by the gastrointestinal tract, the tract has an inner mucosal layer 46, an outer muscularis layer 48, and connective fibers 64 (FIG. 8) connecting the two layers.

The distal end of the endoscopic injection needle 50 is advanced through a first working channel 40 of the endoscope 42 and inserted into a target tissue, such as a lesion 52. The endoscopic injection needle 50 is used to inject a lifting agent, such as saline, under the target tissue 52 to lift the target tissue and thereby define a perimeter 54 about the target tissue in a first wall 55 of the gastrointestinal tract. The injection needle 50 is then removed from the first working channel of the endoscope.

Turning to FIG. 4, the distal end of the electrosurgical instrument 60 is then advanced through the first working channel 40 of the endoscope 42 and operated to cut through the mucosal tissue 46 along the defined perimeter 54 of the target tissue. The electrosurgical instrument is then preferably removed from the first working channel 40, or preferably at least from the visual field.

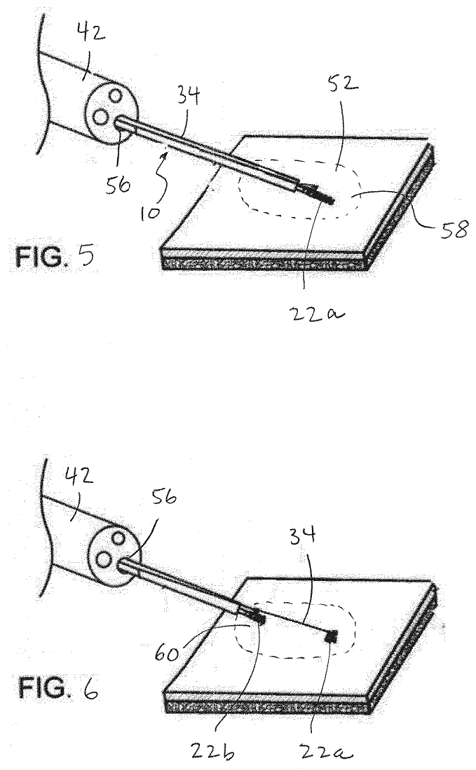

Referring now to FIGS. 1, 2, and 5, the suture anchor deployment system 10 is readied with a suture anchor 22 mounted at the delivery member 20 of the deployment system, and a suture 34 fixed to the eyelet 28 of the suture anchor. The distal end of the suture anchor deployment system is then advanced through a second working channel 56 of the endoscope 42. Optionally, where the endoscope has a single working channel, the electrosurgical instrument can be withdrawn, while the suture anchor deployment system is advanced into the sole working channel. The distal end of the suture anchor deployment system 10 is maneuvered toward a distal portion of the target tissue 52, preferably adjacent the proximal margin 58 of the lesion, and operated to apply a first suture anchor 22a thereat. The length of suture 34 fixed to the first suture anchor 22a extends out of the gastrointestinal tract and the endoscope 42.

The suture anchor deployment system 10 is then withdrawn from the second working channel 56, the suture 34 is thread through the suture eyelet 28 of a second suture anchor 22b, and the second suture anchor 22b is mounted at the distal end of the suture anchor deployment system 10.

Tuning to FIG. 6, the suture anchor deployment system 10 is again advanced through the second working channel 56 and advanced toward a proximal portion of the target tissue, preferably adjacent the proximal margin 60 of the lesion, and operated to apply the second anchor 22b thereat.

The suture anchor deployment system 10 is then withdrawn from the second working channel 56, the suture 34 is thread through the suture eyelet 28 of a third suture anchor 22c, and the third suture anchor 22c is mounted at the distal end of the suture anchor deployment system 10.

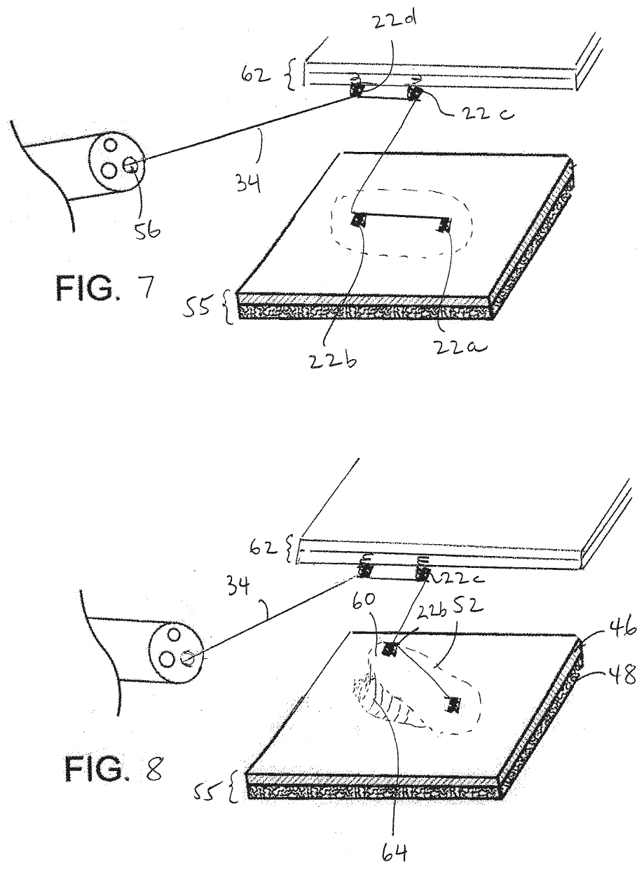

Referring to FIG. 7, the suture anchor deployment system 10 is again advanced through the second working channel 56 and this time extended toward a second wall 62 of the gastrointestinal tract located opposite the first wall 55. One or more additional suture anchors 22c, 22d are inserted in the second wall. After the last suture anchor is implanted, the suture anchor deployment system 10 is preferably withdrawn from the second working channel 56 of the endoscope. If the electrosurgical instrument 50 is not still within a working channel of the endoscope, it is then reinserted to adjacent the target tissue (not shown).

Referring to FIG. 8, the suture is then tensioned. Tension may be applied by pulling on the suture or by insufflating the body cavity to increase the distance between the first and second walls 55, 62. Such tension causes the suture 34, fixed to the first anchor but movable relative to the other anchors, to be drawn relative to the second and third anchors 22b, 22c and thereby apply traction between the second wall 62 and the relatively proximal portion of the target tissue, i.e., the proximal margin 60 of the lesion 52. Thus, as the suture moves through the eyelets of the suture anchors 22b, 22c, a pulley system is formed, with suture anchor 22c functioning as a pulley that assists in lifting the lesion and controlling tissue traction. This stretches the connective fibers 64 between the mucosal layer 46 and the muscularis layer 48 of the dissected lesion.

In another alternate embodiment of anchor application which utilizes an endoscope with a single working channel, after first using the electrosurgical instrument, the electrosurgical instrument is removed and then the suture anchor deployment system is inserted through the working channel and operated to deploy all tissue anchors. Then, the suture anchor deployment system is removed from the working channel and an electrosurgical instrument is inserted into the working channel alongside the suture. The suture is then tensioned from outside the patient through the working channel alongside the electrosurgical instrument.

In yet another embodiment using an endoscope with a single working channel, the procedure can be carried out as follows: the injection needle is inserted through the working channel and operated to inject at the lesion to create a bleb; then, the injection needle is retracted from the working channel. The suture anchor deployment system is next inserted through the working channel, and then operated to position anchors in the lesion as well as in the opposing wall. Then, the suture anchor deployment system is removed from the endoscope, leaving the suture through the working channel. Next, the endoscope is removed from patient leaving the suture extending from the anchors and exiting the natural orifice. Then, the endoscope is again inserted through the natural orifice alongside the suture. An electrosurgical instrument can be inserted through the working channel. The suture can be tensioned from outside the patient to manipulate the lesion without interfering with the endoscope or the working channel.

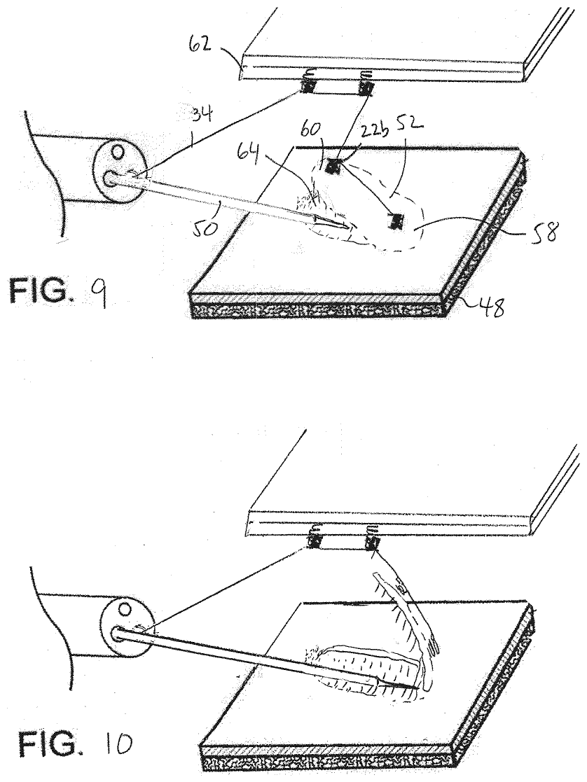

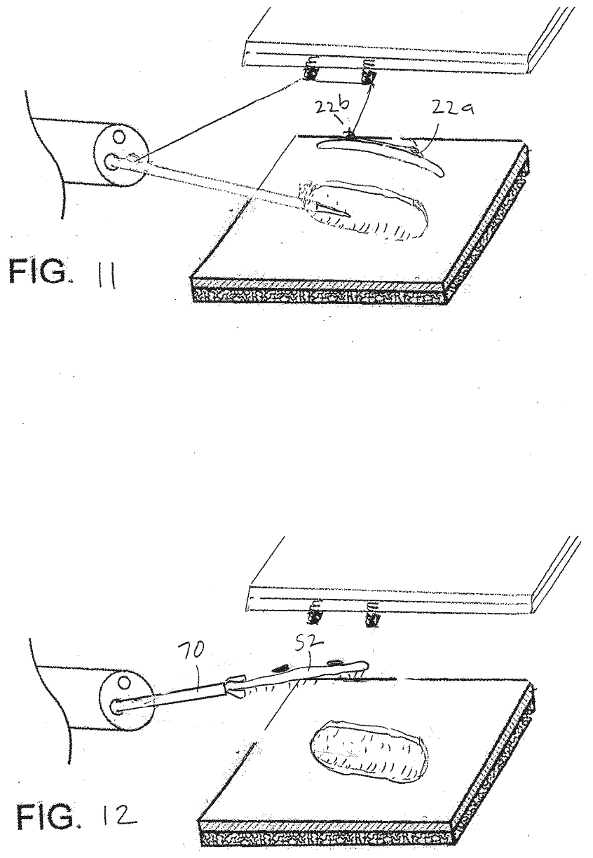

Turning to FIG. 9, the electrosurgical instrument 50 is then operated to cut the stretched connective fibers 64 under the proximal portion 60 of the lesion 52. With tension applied to the suture 34, the second anchor 22b, holding the proximal portion 60, lifts up toward the second wall 62. Further tensioning the suture draws the suture relative to the first 22a and third anchors 22c and thereby applies traction between the second wall 62 and the relatively distal portion 58 of the target tissue, i.e., distal margin of the lesion, to stretch connective fibers 64 between the mucosal layer and the muscularis layer thereat. Referring to FIG. 10, the electrosurgical instrument 50 is again operated to cut the stretched connective fibers 64 under the distal margin 58 of the lesion to separate the lesion 52 from the muscularis layer 48. The process may be repeated in intermediate steps before the lesion is completely separated, as shown in FIG. 11.

The dissected lesion is attached to suture with at least two anchors 22a, 22b. A scissors or other blade may be used to cut the suture, and a tissue grasper 70 may be used retrieve the lesion from the patient. The dissected lesion is retrieved en masse for histological examination.

The method permits tension to be applied to a lesion even after the connective fibers under the proximal margin of the lesion are cut. The method also permits identifying and applying tension at multiple locations during removal of the lesion. This facilitates maintaining high visibility at the operative location during the procedure and permits clean and efficient removal of the entire lesion.

In addition, the system and method can be used for tissue retrieval during colonic resections. In such a method, the above method is used to dissect multiple locations along the colon. Thus, the procedure allows securement of multiple polyp specimens in-situ without necessitating retrieval of the scope to extract the each individual specimen. One or more anchors are positioned in a lesion, and the lesion is dissected; another anchor is placed in the next lesion; and the next lesion is dissected. The process continues until all intended lesions are dissected. Once all intended lesions have been dissected, the scope is removed and suture is retrieved with all tissue specimens attached to the end of the suture, with lesion specimen identified by the order in which the anchors where placed.

There have been described and illustrated herein methods of tissue traction, methods of dissecting a mucosal layer from the muscularis layer in the gastrointestinal tract, and methods of endoscopic tissue dissection of a lesion from a mucosal layer of a gastrointestinal tract of a patient. While particular embodiments of the invention have been described, it is not intended that the invention be limited thereto, as it is intended that the invention be as broad in scope as the art will allow and that the specification be read likewise. Thus, while the method is described with respect to a re-loadable anchor deployment system, the method can also be performed with an anchor deployment system that carriers a plurality preloaded anchors within the deployment system and coupled to the suture and deploys the anchor in sequence and coupled to the suture. In addition, while the term “suture” is referred to throughout for convenience as it is a medical grade material meeting the requirement of the system and procedure, it is appreciated that the material referred to as a suture never extends through tissue and thus does not function like suture in the traditional sense. Thus, the term suture is meant to refer to any tether, string, cable, or wire of suitable flexibility that can function as required for the described system and methods. It will therefore be appreciated by those skilled in the art that yet other modifications could be made to the provided invention without deviating from its scope as claimed.

What is claimed is:

1. A method of dissecting a mucosal layer from a muscularis layer in a gastrointestinal tract, comprising:
a) applying traction to tension connective fibers between the mucosal and muscularis layers; and
b) cutting the tensioned connective fibers.

2. The method of claim 1, further comprising:
incising about a target area of tissue having the mucosal and muscularis layers; and
removing the target area of the mucosal layer after the tensioned connected fibers are cut.

3. The method of claim 2, wherein the applying traction, cutting, incising and removing are performed endoscopically through a natural orifice.

4. The method of claim 1, wherein the applying traction and cutting are performed endoscopically through a natural orifice.

5. The method of claim 1, wherein applying traction includes tensioning suture attached to the mucosal layer.

6. The method of claim 5, wherein applying traction includes further includes insufflating a portion of the gastrointestinal tract.

7. The method of claim 5, wherein applying traction includes lifting the mucosal layer with an aid of a pulley.

8. A method of endoscopic tissue traction in a natural body cavity, comprising:
a) advancing a flexible endoscopic into the natural body cavity through a natural orifice, the endoscope having a proximal end, a distal end, and a working channel extending from the proximal to the distal end;
b) providing an anchor deployment system advanceable through the working channel and operable to apply a plurality of like anchors into tissue in the natural body cavity;
c) first, using the anchor deployment system to apply a first anchor of the like anchor into a relatively distal portion of a tissue in a first wall in the natural body cavity, a distal end of a length of suture fixed to the first anchor;
d) second, using the anchor deployment system to apply a second anchor of the like anchors into a relatively proximal portion of the tissue in the first wall in the natural body cavity, the length of suture coupled to and displaceable relative to the second anchor;
e) third, using the suture anchor deployment system to apply a third anchor of the like anchors into a second wall opposite the first wall in the natural body cavity, the length of suture coupled to and displaceable relative to the third anchor; and
f) tensioning the suture to draw the suture relative to the second and third anchors and thereby apply traction between the second wall and the relatively proximal portion of the tissue.

9. The method of claim 8, wherein, the suture anchor deployment system includes a proximal handle, an elongate flexible delivery member having proximal and distal ends, the delivery member having a length and diameter sized for passage through the working channel, the proximal end of the delivery member rotatably coupled to the handle, and a suture anchor engaging structure at the distal end of the delivery member.

10. The method of claim 8, wherein the anchors each include a tissue-engaging helical coil.

11. The method of claim 10, wherein the anchors each include a suture eyelet rotatable relative to the helical coil, and the length of suture is coupled to the suture eyelet.

12. The method of claim 8, wherein the suture extends through the endoscope.

13. The method of claim 8, wherein the distal portion is a distal margin of a lesion, and the proximal portion is a proximal margin of the lesion.

14. The method of claim 8, further comprising:
before tensioning, using the suture anchor deployment system to apply a fourth anchor of the like anchors into the second wall in the natural body cavity, the length of suture displaceable relative to the fourth anchor.

15. A method of endoscopic tissue dissection of a lesion from a mucosal layer of a gastrointestinal tract of a patient, the mucosal layer connected to an underlying muscularis layer with connective fibers, the method comprising:
a) injecting a lifting agent under the lesion to lift the lesion and define a perimeter of the lesion in a first wall of the gastrointestinal tract;
b) dissecting the lesion about the defined perimeter;
c) providing a suture anchor deployment system operable to apply a plurality of like anchors;
d) first, using the suture anchor deployment system to apply a first anchor of the like anchors into a relatively distal portion of the lesion in the first wall of the gastrointestinal tract, a distal end of a length of suture fixed to the first anchor;
e) second, using the suture anchor deployment system to apply a second anchor of the like anchors into a relatively proximal portion of the lesion in the first wall, the length of suture coupled to and displaceable relative to the second anchor;
f) third, using the suture anchor deployment system to apply a third anchor of the like anchors into a second wall of the gastrointestinal tract located opposite the first wall, the length of suture coupled to and displaceable relative to the third anchor;
g) tensioning the suture to draw the suture relative to the second and third anchors and thereby apply traction between the second wall and the relatively proximal portion of the lesion to stretch the connective fibers between the mucosal layer and the muscularis layer of the dissected lesion.
h) cutting the stretched connective fibers under the proximal portion of the lesion;
i) further tensioning the suture to draw the suture relative to the first and third anchors and thereby apply traction between the second wall and the relatively distal portion of the lesion to stretch connective fibers between the mucosal layer and the muscularis layer; and
j) cutting the stretched connective fibers under the distal portion of the lesion to completely separate the lesion from the muscularis layer.

16. The method according to claim 15, further comprising retrieving the separated lesion from the gastrointestinal tract.

17. The method according to claim 15, further comprising:
advancing an endoscope into the gastrointestinal tract through a natural orifice, the endoscope having a proximal end, a distal end, and at least one working channel extending from the proximal end to the distal end,
advancing a needle instrument through the at least one working channel,
wherein the needle instrument is used to inject the lifting agent.

18. The method according to claim 15, further comprising:
advancing an endoscope into the gastrointestinal tract through a natural orifice, the endoscope having a proximal end, a distal end, and at least one working channel extending from the proximal end to the distal end,
advancing an electrosurgical knife through the at least one working channel,
wherein the electrosurgical knife is used to dissect the lesion and cut the connective fibers.

19. The method according to claim 15, further comprising:
advancing an endoscope into the gastrointestinal tract through a natural orifice, the endoscope having a proximal end, a distal end, and at least one working channel extending from the proximal end to the distal end,
wherein the suture anchor deployment system includes a proximal handle, an elongate flexible delivery member having proximal and distal ends, the delivery member having a length and diameter sized for passage through the at least one working channel, the proximal end of the delivery member rotatably coupled to the handle, and a suture anchor engaging structure at the distal end of the delivery member.

20. The method of claim 19, wherein the suture extends a working channel of the endoscope.

21. The method of claim 15, wherein the anchors each include a tissue-engaging helical coil.

22. The method of claim 21, wherein the anchors each include a suture eyelet rotatable relative to the helical coil, and the length of suture is coupled to the suture eyelet.

23. The method of claim 15, wherein the distal portion is a distal margin of a lesion, and the proximal portion is a proximal margin of the lesion.

24. The method of claim 15, further comprising:
before tensioning, using the suture anchor deployment system to apply a fourth anchor of the like anchors into the second wall, the length of suture displaceable relative to the fourth anchor.

25. A method of endoscopic lesion removal from the gastrointestinal tract through a natural orifice, the method comprising:
inserting a first helical tissue anchor attached to a suture through the natural orifice and into the gastrointestinal tract;
engaging the first helical tissue anchor to a first lesion in the gastrointestinal tract;
dissecting the first lesion; and
retracting the suture to remove the first helical tissue anchor and engaged first lesion from natural orifice.

26. The method of claim 25, further comprising:
inserting a second helical tissue anchor attached to the suture through the natural orifice and into the gastrointestinal tract; and
engaging the second helical tissue anchor to a second lesion in the gastrointestinal tract;
dissecting the second lesion,
wherein the retracting the suture also removes the second helical tissue anchor and engaged second lesion from the natural orifice.
